(19) United States
(12) Patent Application Publication
(10) Pub. No.: US 2014/0135324 A1
(43) Pub. Date: May 15, 2014

(54) Rapamycin Derivative or an Impdh Inhibitor for Treating Polycystic Kidney Disease

(71) Applicants: Lothar Färber, Heroldsberg (DE); Harald GSCHAIDMEIER, Nurnberg (DE)

(72) Inventors: Lothar Färber, Heroldsberg (DE); Harald GSCHAIDMEIER, Nurnberg (DE)

(21) Appl. No.: 14/162,103

(22) Filed: Jan. 23, 2014

Related U.S. Application Data
(63) Continuation of application No. 13/463,461, filed on May 3, 2012.
Application No. 13/463,461 is a continuation of application No. 13/029,741, filed on Feb. 17, 2011, now abandoned.
Application No. 13/029,741 is a continuation of application No. 12/093,507, filed on May 13, 2008, now abandoned.

(30) Foreign Application Priority Data
Nov. 21, 2005 (GB) 0523659.1

(51) Int. Cl. A61K 31/5377; A61K 31/439

(52) U.S. Cl. CPC A61K 31/5377; A61K 31/439
USPC 514/233.5; 514/291

(57) ABSTRACT

A method for treating polycystic kidney disease, comprising administering to a subject in need thereof a therapeutical effective amount of an inosine-5′-monophosphate dehydrogenase inhibitor or a rapamycin derivative.

The present invention relates to agents for treating polycystic kidney disease, more specifically to a new use of rapamycin derivatives and inosine-5′monophosphate dehydrogenase inhibitors.

Rapamycin is a known macrolide antibiotic produced by Streptomyces hygroscopicus of formula

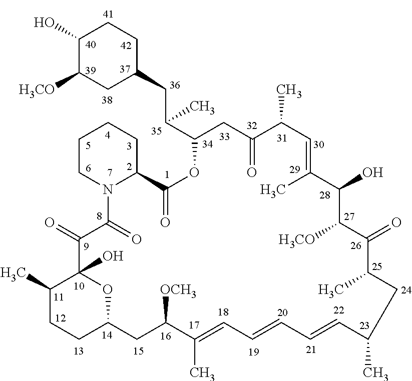

Rapamycin derivatives include substituted rapamycin, e.g. rapamycin substituted in position 40 and/or 16 and/or 32.

Examples of rapamycin derivatives include 40-O-alkyl-rapamycin derivatives, e.g. 40-O-hydroxyalkyl-rapamycin derivatives, for example 40-O-(2-hydroxy)-ethyl-rapamycin (everolimus),

rapamycin derivatives which are substituted in 40 position by heterocyclyl, e.g. 40-epi-(tetrazolyl)-rapamycin (also known as ABT578),

32-deoxo-rapamycin derivatives and 32-hydroxy-rapamycin derivatives, such as 32-deoxorapamycin,

16-O-substituted rapamycin derivatives such as 16-pent-2-ynyloxy-32-deoxorapamycin, 16-pent-2-ynyloxy-32(S or R)-dihydro-rapamycin, or 16-pent-2-ynyloxy-32(S or R)-dihydro-40-O-(2-hydroxyethyl)-rapamycin,

rapamycin derivatives which are acylated at the oxygen in position 40, e.g. 40-[3-hydroxy-2-(hydroxy-methyl)-2-methylpropanoate]-rapamycin (also known as CCI779 or temsirolimus), rapamycin derivatives (also sometimes designated as rapalogs) as disclosed in WO9802441 or WO0114387, e.g. including AP23573, such as 40-O-dimethyiphosphinyl-rapamycin, compounds disclosed under the name biolimus (biolimus A9), including 40-O-(2-ethoxy)ethyl-rapamycin, and compounds disclosed under the name TAFA-93, AP23464, AP23675 or AP23841; or

rapamycin derivatives as e.g. disclosed in WO2004101583, WO9205179, WO9402136, WO9402385 and WO9613273.

Preferred rapamycin derivatives include

40-O-(2-hydroxyethyl)-rapamycin, and/or

32-deoxorapamycin, and/or

16-pent-2-ynyloxy-32-deoxorapamycin, and/or

16-pent-2-ynyloxy-32 (S or R)-dihydro-rapamycin, and/or

16-pent-2-ynyloxy-32 (S or R)-dihydro-40-O-(2-hydroxyethyl)-rapamycin, and/or

40-[3-hydroxy-2-(hydroxy-methyl)-2-methylpropanoate]-rapamycin (also known as CCI779) and/or

40-epi-(tetrazolyl)-rapamycin (also known as ABT578), and/or

the so-called rapalogs, e. g. as disclosed in WO9802441, WO0114387 and WO0364383, AP23573, AP23464; AP23675 or AP23841, e.g. AP23573, and/or

compounds disclosed under the name TAFA-93, and/or

compounds disclosed under the name biolimus.

More preferably a rapamycin derivative is selected from the group consisting of

40-O-(2-hydroxyethyl)-rapamycin (also known as everolimus), and/or

32-deoxorapamycin, and/or

16-pent-2-ynyloxy-32-deoxorapamycin, and/or

16-pent-2-ynyloxy-32 (S or R) -dihydro-rapamycin, and/or

16-pent-2-ynyloxy-32 (S or R)-dihydro-40-O-(2-hydroxyethyl)-rapamycin, and/or

40-[3-hydroxy-2-(hydroxy-methyl)-2-methylpropanoate]-rapamycin (also known as CC1779 or temsirolimus) and/or

40-epi-(tetrazolyl)-rapamycin (also known as ABT578), and/or

AP23573,

such as 40-O-(2-hydroxyethyl)-rapamycin.

Inosine-5′-monophosphate dehydrogenase (IMPDH) is an enzyme involved in the de novo synthesis of guanosine nucleotides. IMPDH catalyzes the NAD-dependent oxidation of inosine-5′-monophosphate (IMP) to xanthosine-5′-monophosphate (XMP) (Jackson R. C. et. al., Nature, 256:331-333 (1975)).

IMPDH inhibitors are known and e.g. include ribavirin, tiazofurin, Vertex VX148, VX-497, VX944, merimepodib, benzamide riboside, mycophenolic acid and salts, mycophenolate mofetil, also known under the trade name Cellcept®, e.g. in combination with standard or PEG-interferon alpha-2a with or without ribavirin; Avalon AVN944.

Mycophenolic acid, also referred to herein as MPA, was first isolated in 1896. Suitable MPA salts include cationic salts, e. g. alkali metal salts, especially the sodium salt, e. g. mono or di-sodium salt, preferably mono-sodium salt. Prodrugs of MPA include e. g. physiologically hydrolysable esters of MPA, e. g. as disclosed in U.S. Pat. No. 4,753,935 such as the morpholinoethyl ester, also known as mycophenolate mofetil (MMF). Preferred is a sodium mycophenolate salt, such as myciophenolate sodium. Mycophenolate salts when enteric coated or adapted to be released in the upper part of the intestines lead to effective, well-tolerated, pharmaceuticals particularly for immuno-suppressive indications, e. g. treatment or prevention of cell, tissue or organ allograft rejection.

Polycystic kidney disease (PKD) is a genetic disorder characterized by the growth of numerous cysts in the kidneys. The cysts are filled with fluid. PKD cysts can slowly replace much of the mass of the kidneys, reducing kidney function and leading to kidney failure. The kidneys are two organs, each about the size of a fist, located in the upper part of a person's abdomen, toward the back. The kidneys filter wastes from the blood to form urine. They also regulate amounts of certain vital substances in the body.

When PKD causes kidneys to fail which usually happens after many years the patient requires dialysis or kidney transplantation. About one-half of people with the major type of PKD progress to kidney failure, also called end-stage renal disease (ESRD).

PKD can cause cysts in the liver and problems in other organs, such as the heart and blood vessels in the brain. These complications help doctors distinguish PKD from the usually harmless “simple” cysts that often form in the kidneys in later years of life.

In the United States, about 500,000 people have PKD, and it is the fourth leading cause of kidney failure. Medical professionals describe two major inherited forms of PKD and a noninherited form, namely adult dominant PKD (ADPKD), Autosomal recessive polycystic kidney disease (ARPKD), and acquired cystic kidney disease (ACKD).

Adult PKD (APKD) is the most common inherited form. Adult PKD (ADPKD) is an autosomal dominant disorder (ADPKD). ADPKD is one of the most common monogenic hereditary diseases. The disease is considered to be a systemic disorder, characterized by cyst formation in the ductal organs such as kidney liver, and pancreas, as well as by gastrointestinal, cardiovascular, and musculoskeletal abnormalities, including colonic diverticulitis, berry aneurysms, hernias, and mitral valve prolapse (Gabow et at, Adv. Nephrol, 18:19-32, 1989; Gabow, New Eng. J. Med., 329:332-342, 1993). The most prevalent and obvious symptom of APKD, however, is the formation of kidney cysts, which result in grossly enlarged kidneys and a decrease in renal-concentrating ability.

ADPKD-associated renal cysts may enlarge to contain several liters of fluid and the kidneys usually enlarge progressively causing pain. Other abnormalities such as pain, hematuria, renal and urinary infection, renal tumors, salt and water imbalance, hypertension and other endocrine abnormalities result from the renal defect. The disease is characterized by the proliferation of epithelial cells, formation of renal cysts, liver cysts, intracranial aneurysm, and progression of renal insufficiency. urinary tract infections. Symptoms include hematuria (blood in the urine), liver and pancreatic cysts, abnormal heart valves, high blood pressure, kidney stones, aneurysms (bulges in the walls of blood vessels) in the brain and diverticulosis (small sacs on the colon)

In approximately half of APKD patients, the disease progresses to end-stage renal disease; accordingly, APKD is responsible for 4-8% of the renal dialysis and transplantation cases in the United States and Europe (Proc. European Dialysis and Transplant Assn., Robinson and Hawkins, eds., 17:20, 1981). Thus, there is a need in the art for therapeutic tools to reduce the incidence and severity of this disease.

ADPKD is associated with pain, urinary tract infection and high blood pressure. At moment only the symptoms can be treated in view of lack of successful disease treatment.

Autosomal recessive polycystic kidney disease (ARPKD) is an important renal cause of death in the perinatal period and of childhood renal failure. Neonatal disease presentation is typical, and characterized by greatly enlarged kidneys due to fusiform dilation of collecting ducts; congenital hepatic fibrosis is often a major complication in older patients. Progress toward understanding this complex disorder has recently been made by the identification of the disease-causing gene, PKHD1, in chromosome region 6pI2. PKHD1 is a very large gene (470 kb) containing 67 exons and an open reading frame (ORE) of 12,222 bp. PI(HD1 has so a tissue-specific expression pattern with the highest levels in fetal and adult kidney and lower levels in liver, pancreas and lung. The murine otholog, Pilldl, has recently been described.

Children with autosomal recessive PKD experience high blood pressure, urinary tract infections, and frequent urination. The disease usually affects the liver, spleen, and pancreas, and causes low blood-cell counts, varicose veins, and hemorrhoids. Because kidney function is crucial for early physical development, children with autosomal recessive PKD are usually smaller than average size. Treatment of the symptoms includes controlling blood pressure, controlling murinary tract infection and sometimes administering growth hormones.

ACKD develops in kidneys with long-term damage and bad scarring, so it often is associated with dialysis and end-stage renal disease. About 90 percent of people on dialysis for 5 years develop ACKD. People with ACKD can have any underlying kidney disease, such as glomerulonephritis or kidney disease of diabetes.

Symptoms include that the cysts of ACKD may bleed. Kidney tumors, including kidney (renal) cancer, can develop in people with ACKD. Although renal cancer is rare, it occurs at least twice as often in ACKD patients as in the general population.

It was now surprisingly found that inosine-5′-monophosphate dehydrogenase inhibitors and rapamycin derivatives may be used for the treatment of polycystic kidney disease.

In accordance with the particular findings the present invention provides:

1.1 A method for treating polycystic kidney disease, comprising administering to a subject in need thereof a therapeutically effective amount of a rapamycin derivative or an inosine-5′-monophosphate dehydrogenase inhibitor.

1.2 A method for inhibiting growth of a cyst in the kidney, which cyst is associated with polycystic kidney disease, comprising administering to a subject in need thereof a therapeutically effective amount of a rapamycin derivative or an inosine-5′-monophosphate dehydrogenase inhibitor.

1.3 A method for inhibiting or controlling polycystic kidney disease, comprising administering to a subject in need thereof a therapeutically effective amount of a rapamycin derivative or an inosine-5′-monophosphate dehydrogenase inhibitor.

1.4 A method for inducing polycystic kidney disease regression, e. g. inducing cyst mass reduction, comprising administering to a subject in need thereof a therapeutically effective amount of a rapamycin derivative or an inosine-5′-monophosphate dehydrogenase inhibitor.

1.5 A method for the treatment of a disorder associated with polycystic kidney disease, comprising administering to a subject in need thereof a therapeutically effective amount of a rapamycin derivative or an inosine-5′-monophosphate dehydrogenase inhibitor.

1.6 The use of a rapamycin derivative or an inosine-5′-monophosphate dehydrogenase inhibitor for the manufacture of a medicament for use in any method of 1.1 to 1.5 above.

1.7 A pharmaceutical composition comprising a rapamycin derivative or an inosine-5′-monophosphate dehydrogenase inhibitor in association with at least one pharmaceutically acceptable excipient, e.g. appropriate carrier and/or diluent, e.g. including fillers, binders, disintegrants, flow conditioners, lubricants, sugars or sweeteners, fragrances, preservatives, stabilizers, wetting agents and/or emulsifiers, solubilizers, salts for regulating osmotic pressure and/or buffers; for use in any method or use of 1.1 to 1.5 above.

Polycystic kidney disease as indicated herein includes adult polycystic kidney disease (ADKP) which is an autosomal dominant form of PKD (ADPKD), autosomal recessive polycystic kidney disease (ARPKD) and acquired cystic kidney disease (ACKD).

An inosine-5′-monophosphate dehydrogenase inhibitor or a rapamycin derivative is herein designated as “compound(s) of (according to) the present invention” with the proviso that a compound of the present invention is either an inosine-5′-monophosphate dehydrogenase inhibitor or a rapamycin derivative.

A compound of the present invention may be used, e.g. in any method of 1.1 to 1.6 as described herein alone or in combination with one or more, at least one, second drug substance.

In other aspects the present invention provides

2.1 A combination of a compound of the present invention with at least one second drug substance, e.g. for any use as indicated under 1.1 to 1.5 above.

2.2 A pharmaceutical combination comprising a compound of the present invention in combination with at least one second drug substance, e.g. for any use as indicated under 1.1 to 1.5 above.

2.3 A pharmaceutical composition comprising a compound of the present invention in combination with at least one second drug substance and one or more pharmaceutically acceptable excipient(s), e.g. for any use as indicated under 1.1 to 1.5 above.

2.4 The use of a compound of the present invention for the manufacture of a medicament for use in combination with a second drug substance, e.g. for any use as indicated under 1.1 to 1.5 above.

2.5 Any method of 1.1 to 1.5 above comprising co-administering, concomitantly or in sequence, a therapeutically effective amount of a compound of the present invention and at least one second drug substance, e.g. in the form of a pharmaceutical combination or composition.

2.6 A compound of the present invention in combination with at least one second drug substance for use in the preparation of a medicament, e.g. for use in any method of 1.1 to 1.5 above.

Combinations include fixed combinations, in which a compound of the present invention and at least one second drug substance are in the same formulation; kits, in which a compound of the present invention and at least one second drug substance in separate formulations are provided in the same package, e.g. with instruction for co-administration; and free combinations in which a compound of the present invention and at least one second drug substance are packaged separately, but instruction for concomitant or sequential administration are given.

In another aspect the present invention provides

2.7 A pharmaceutical package comprising a first drug substance which is a compound of the present invention and at least one second drug substance, beside instructions for combined administration;

2.8 A pharmaceutical package comprising a compound of the present invention beside instructions for combined administration with at least one second drug substance;

2.9 A pharmaceutical package comprising at least one second drug substance beside instructions for combined administration with a compound of the present invention;

e.g. for use in any method of 1.1 to 1.5 above.

Treatment with combinations according to the present invention may provide improvements compared with single treatment.

In another aspect the present invention provides

2.10 A pharmaceutical combination comprising an amount of a compound of the present invention and an amount of a second drug substance, wherein the amounts are appropriate to produce a synergistic therapeutic effect. 2.11-A method for improving the therapeutic utility of a a compound of the present invention comprising co-administering, e.g. concomitantly or in sequence, a therapeutically effective amount of a compound of the present invention and a second drug substance.

2.12 A method for improving the therapeutic utility of a second drug substance comprising co-administering, e.g. concomitantly or in sequence, a therapeutically effective amount of an compound of the present invention and a second drug substance.

e.g. for use in any method of 1.1 to 1.6 above.

In a pharmaceutical combination or in a method of 2.11 to 2.12 above the activity of a compound of the present invention or the activity of a second drug substance may be increased compared with single treatment, e.g. combined treatment may result in synergistic effects or may overcome resistance against a compound of the present invention or a chemotherapeutic agent, e.g. when used in any method according to 1.1 to 1.6 as described above.

A (pharmaceutical) combination, e.g. composition, as indicated under 2.1 to 2.12 comprises

a) a first agent which is a compound of the present invention, and

b) a second drug substance as a co-agent which is a chemotherapeutic agent, e. g. as defined hereinafter or hereinbefore

with the proviso that a first agent is either a rapamycin derivative or an inosine-5′-monophosphate dehydrogenase inhibitor and, in case that the first agent is a rapamycin derivative, a second drug substance includes an inosine-5′-monophosphate dehydrogenase inhibitor; and vice versa.

Treatment of disorders (diseases) according to the present invention includes prophylaxis (prevention).

For such treatment, the appropriate dosage will, of course, vary depending upon, for example, the chemical nature and the pharmacokinetic data of a compound used, the individual host, the mode of administration and the nature and severity of the conditions being treated. However, in general, for satisfactory results in larger mammals, for example humans, an indicated daily dosage includes a range
from about 0.0001 g to about 1.5 g, such as 0.001 g to 1.5 g;
from about 0.001 mg/kg body weight to about 20 mg/kg body weight, such as 0.01 mg/kg body weight to 20 mg/kg body weight,
   for example administered in divided doses up to four times a day.

In a method, for use or in a combination, pharmaceutical combination or pharmaceutical composition provided by the present invention a rapamycin derivative, may be administered as appropriate, e.g. in dosages which are known for rapamycin derivatives, by any administration route, e.g. enterally, e.g. orally, or parenterally. E.g. everolimus may be administered, e.g. orally, in dosages from 0.1 mg up to 15 mg, such as 0.1 mg to 10 mg. e.g. 0.1 mg, 0.25 mg, 0.5 mg, 0.75 mg, 1 mg, 2.5 mg, 5 mg, or 10 mg, more preferably from 0.5 mg to 10 mg, e.g. in the form of (dispersible) tablets; e.g. comprising everolimus in the form of a solid dispersion; e.g. a weekly dosage may include up to 70 mg, e.g. 10 to 70, such as 30 to 50 mg, depending on the disease being treated. E.g. temsirolimus may be administered parenterally in similar dosage ranges.

In a method, for use or in a combination, pharmaceutical combination or pharmaceutical composition provided by the present invention an IMPDH inhibitor may be administered as appropriate, e.g. according to product description. E.g. mycophenolate sodium may be e.g. administered orally, e.g. in the form of tablets. Enteric coated mycophenalete in the form of a sodium salt is also known under the trade name Myfortic®. Dosage ranges e.g. include 50 mg to 1500 mg, such as 100 mg to 1000 mg, e.g. 150 mg to 500 mg, such as 180 mg, 360 mg.

A second drug substance may be administered in combination therapy as appropriate, e.g. according to a method as conventional, e.g. analogously to administration indications given for a specified drug for single treatment.

A second drug substance according to the present invention may be administered by any conventional route, for example enterally, e.g. including nasal, buccal, rectal, oral, administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including epicutaneous, intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye); or via medical devices, e.g. for local delivery, e.g. stents; e.g. in form of coated or uncoated tablets, capsules, (injectable) solutions, infusion solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

A second drug substance according to the present invention may be administered in the form of a pharmaceutically acceptable salt, or in free form; optionally in the form of a solvate. Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

Pharmaceutical compositions comprising a combination of the present invention and pharmaceutical compositions comprising a second drug substance as described herein, may be provided as appropriate, e.g. according, e.g. analogously, to a method as conventional, or as described herein for a pharmaceutical composition of the present invention.

By the term “second drug substance” as used herein is meant

a) in case that the first drug substance is a rapamycin derivative,
a rapamycin derivative other than the first drug substance, or
a chemotherapeutic agent other than a rapamycin derivative, or

b) in case that the first drug substance is an inosine-5′-monophosphate dehydrogenase inhibitor
an inosine-5′-monophosphate dehydrogenase inhibitor other than the first drug substance,
a chemotherapeutic agent other than an inosine-5′-monophosphate dehydrogenase inhibitor.

Such second drug substance preferably is a drug substance which is therapeutically useful for PKD and/or PKD symptom treatment and optionally includes, e.g. for PKD symptom treatment e.g. antibiotics, blood pressure lowering agents, pain killers. Such second drug substance additionally includes rapamycin.

Preferably a second drug substance as used herein includes in case that the first drug substance is a rapamycin derivative,
an inosine-5′-monophosphate dehydrogenase inhibitor,
a pain killer,
an antibiotic,
a blood lowering agent.

Preferred combinations include
a rapamycin derivative in combination with a pain killer, antibiotic, and/or blood lowering agent;
an inosine-5′-monophosphate dehydrogenase inhibitor in combination with a pain killer, antibiotic, and/or blood lowering agent;
a rapamycin derivative in combination with an inosine-5′-monophosphate dehydrogenase inhibitor, optionally further comprising a pain killer, antibiotic, and/or blood lowering agent.

In another aspect the present invention provides

3.1 Any method, combination, pharmaceutical combination, pharmaceutical composition or use as indicated under 1.1 to 1.7 and 2.1 to 2.13 above wherein a compound of the present invention is selected from a rapamycin derivative, e.g.
40-O-(2-hydroxyethyl)-rapamycin (also known as everolimus), and/or
32-deoxorapamycin, and/or
16-pent-2-ynyloxy-32-deoxorapamycin, and/or
16-pent-2-ynyloxy-32 (S or R)-dihydro-rapamycin, and/or
16-pent-2-ynyloxy-32 (S orR)-dihydro-40-O-(2-hydroxyethyl)-rapamycin, and/or
40-[3-hydroxy-2-(hydroxy-methyl)-2-methylpropanoate]-rapamycin (also known as CC1779) and/or
40-epi-(tetrazolyl)-rapamycin (also known as ABT578), and/or
the so-called rapalogs, e. g. as disclosed in WO9802441, WO0114387 and WO0364383, AP23573, AP23464, AP23675 or AP23841, e.g. AP23573, and/or
compounds disclosed under the name TAFA-93, and/or
compounds disclosed under the name biolimus;
e.g. 40-O-(2-hydroxyethyl)-rapamycin (herein also designated as “compound A”).

3.2 Any method, combination, pharmaceutical combination, pharmaceutical composition or use as indicated under 1.1 to 1.7 and 2.1 to 2.13 above wherein a compound of the present invention is selected from an inosine-5′-monophosphate dehydrogenase inhibitor, e.g.
ribavirin, tiazofurin, Vertex VX148, VX-497, VX944, merimepodib, benzamide riboside, mycophenolic acid, a mycophenolate salt, mycophenolate sodium, mycophenolate mofetil, mycophenolate mofetil in combination with standard or PEG-interferon alpha-2a with or without ribavirin or AVN944, e.g.
e.g. mycophenolate sodium, e.g. as marketed under the trade name MYFORTIC®.

In a preferred aspect the present invention provides any method, combination, pharmaceutical combination, pharmaceutical composition, or use as indicated under 1.1 to 1.7 and 2.1 to 2.13 above for the treatment of adult dominant polycistic kidney disease.

In another preferred aspect the present invention provides any method, combination, pharmaceutical combination, pharmaceutical composition, or use as indicated under 1.1 to 1.7 and 2.1 to 2.13 above for the treatment of autosomal recessive polycystic kidney disease

In another preferred aspect the present invention provides any method, combination, pharmaceutical combination, pharmaceutical composition, or use as indicated under 1.1 to 1.7 and 2.1 to 2.13 above for the treatment of acquired cystic kidney disease.

Antibiotics, pain killers and/or blood pressure lowering agents are known or may be provided as appropriate.

In each case where citations of patent applications or scientific publications are given, the subject-matter relating to the compounds is hereby incorporated into the present application by reference, e.g. comprised are likewise the pharmaceutical acceptable salts thereof, the corresponding racemates, diastereoisomers, enantiomers, tautomers as well as the corresponding crystal modifications of above disclosed compounds where present, e. g. solvates, hydrates and polymorphs, which are disclosed therein. The compounds used as active ingredients in the combinations of the invention may be prepared and administered as described in the cited documents or in the product description, respectively. Also within the scope of this invention is the combination of more than two separate active ingredients as set forth above, i. e. a pharmaceutical combination within the scope of this invention could include three active ingredients or more. Further, both the first agent and the co-agent are not the identical ingredient.

The structure of the drug substances identified by code numbers, generic or trade names may be taken from the Internet, actual edition of the standard compendium “The Merck Index” or from databases, e.g., Patents International, e.g., IMS World Publications, or the publications mentioned above and below. The corresponding content thereof is hereby incorporated by reference.

Utility of a compound of the present invention for treating PKD can be shown in a rat model of PKD, e.g. such as a known rat model. Rapamycin derivatives and inosine-5′-monophosphate-dehydrogenase inhibitors appear to be particularly suitable in such rat model.

Clinical trial studies are in preparation.

1. A method for treating polycystic kidney disease, comprising administering to a subject in need thereof a therapeutical effective amount of a rapamycin derivative or an inosine-5′-monophosphate dehydrogenase inhibitor.

2. A method for inhibiting growth of a cyst in the kidney, which cyst is associated with polycystic kidney disease, comprising administering to a subject in need thereof a therapeutically effective amount of a rapamycin derivative or an inosine-5′-monophosphate dehydrogenase inhibitor.

3. A method for inhibiting or controlling polysystic kidney disease, comprising administering to a subject in need thereof a therapeutically effective amount of a rapamycin derivative or an inosine-5′-monophosphate dehydrogenase inhibitor.

4. A method for inducing polycystic kidney disease regression, comprising administering to a subject in need thereof a therapeutically effective amount of a rapamycin derivative or an inosine-5′-monophosphate dehydrogenase inhibitor.

5. A method for the treatment of a disorder associated with polycystic kidney disease, comprising administering to a subject in need thereof a therapeutically effective amount of a rapamycin derivative or an inosine-5′-monophosphate dehydrogenase inhibitor.

6. The method according to claim 1, comprising administering in addition a therapeutically effective amount of at least one second drug substance.

7. The method according to claim 7, wherein the subject is administered a rapamycin derivative and a second drug substance, wherein said second drug substance is an inosine-5′-monophosphate dehydrogenase inhibitor.

8. The method according to claim 8, wherein the rapamycin derivative is 40-O-(2-hydroxyethyl)-rapamycin.

9. The method according to claim 8, wherein the inosine-5′-monophosphate dehydrogenase inhibitor is mycophenolate sodium.
